(19) United States
(12) Patent Application Publication
(10) Pub. No.: US 2003/0104043 A1
(43) Pub. Date: Jun. 5, 2003

(54) Topical cream for alleviating spider veins

(76) Inventors: Beverly Ann Brown, Dallas, TX (US); Gary Osborn, Dallas, TX (US)

Correspondence Address: Robert C. Klinger, Jackson Walker L.L.P., Suite 600, 2435 North Central Expressway, Richardson, TX, 75080, US

(21) Appl. No.: 09/997,592

(22) Filed: Dec. 3, 2001

(51) Int. Cl. A61K 9/127; A61K 31/30

(52) U.S. Cl. 424/450; 514/499

(57) ABSTRACT

A basic therapeutic unit comprises an aqueous solution of copper associated with a skin- and tissue-penetration enabler. Topical application of the unit to the skin penetrates therethrough and through subjacent tissue so that the copper resides at the sites of spider veins for the alleviation or elimination thereof. The copper solution may be copper gluconate and the enabler may be a liposome. A quantity of these therapeutic units may be suspended in a cream or emollient. Additional substances, such as vasodilators, thickeners, preservatives and dispersants may be encapsulated by or contained in the bilayer of the liposome or may be added to the cream.

### FIELD OF THE INVENTION

The present invention relates to therapeutic substances and methods of making and using same, and, more particularly, to a therapeutic unit which serves as the basis for topical copper-delivery systems that are more effective than other delivery systems in marshalling copper at the site of spider veins, and to methods for making and using these copper-delivery systems.

### BACKGROUND OF THE INVENTION

Copper in a variety of forms has, for some time, been used medically and as a dietary supplement. Copper is present in many foods and in multivitamins and has been cited as a key factor in enzyme activity and production, as well as in the utilization of iron within the human body. Copper is also said to be a component of superoxide dismutase. Copper has been included as an ingredient in various skin creams and ointments. Copper peptides are said to alleviate skin dryness, wrinkles and fine lines and to be otherwise essential for good skin health.

Introducing some forms of copper into the body, such as by ingestion, is said to result in the alleviation or elimination of varicose and spider veins. Lastly, an significantly to the present invention, copper has also been cited as a cofactor in several enzymatic reactions related to metabolism, the cross-linking of collagen and elastin, antioxidant formation and skin pigmentation.

Notwithstanding the foregoing, the effective alleviation or elimination of spider veins has eluded those seeking same. Providing the wherewithal to alleviate or eliminate spider veins is one goal of the present invention.

### SUMMARY OF THE INVENTION

In order to achieve the foregoing goal, the present invention in its broadest aspect contemplates a “basic therapeutic unit”—specifically, copper dissolved in an aqueous solution and associated with a carrier which functions as a tissue-penetration enabler. When the carrier is topically applied to the skin overlying spider veins, it transports the copper through the skin and tissue overlying the damaged or affected spider veins and then onto the walls of the affected subjacent spider veins.

It is postulated that spider veins result from a degradation of the normal cross-linking of the collagen which comprises the walls of these blood vessels that reside close beneath the skin. This degradation permits the vessel walls to “bulge,” stretch or become distended, thereby thinning the vessel walls and permitting the dark venous blood therewithin to become visible through the skin. One cause of the degradation of the cross-linked collagen is thought to be a lack of copper ions at receptor sites in the collagen. Copper apparently has the ability to catalyze or support collagen cross-linking. When the copper is brought to the vicinity of the spider veins by the carrier, the copper is taken up by the empty copper-receptor sites and is thereafter available to act as a co-factor in the formation of new, properly cross-linked collagen which re-configures and increases the tensile strength of the vessel walls to eliminate the thinning thereof and the amelioration or disappearance of spider veins.

In one embodiment, the carrier is a liposome and the copper is in the form of an aqueous solution of copper gluconate encapsulated within the liposome. A liposome is a microscopic, generally spherical pouch, sac or bubble (diameter typically about 100 nm). The spherical pouch is defined by a membrane comprised of layers of lipid (e.g., phospholipid) molecules (the “bilayer”) surrounding an aqueous core. Water soluble substances may be encapsulated in the volume defined by the bilayer. Substances that are not easily dissolved in water may be incorporated into the bilayer itself.

Liposomes are able to pass through and between the cells of the skin and subjacent tissue and into or onto a target. The liposomes are incrementally broken up or “digested” by the tissue between the skin and the spider veins as they pass therethrough. At the situs of the spider veins, therefore, the copper solution is released so that copper ions may fill the empty receptor sites, as set forth above.

In another aspect of the present invention, there is provided a therapeutic formulation which is comprised of a plurality of the copper-containing basic therapeutic units held in suspension in a medium—for example an emollient or cream—which is suitable for topical application to the skin. Contained within the volume defined by the liposome membrane along with the encapsulated copper—or incorporated into the bilayer itself—may be other therapeutic substances intended to effect benefits in addition to, or to enhance or supplement the action of, the copper. Similarly, additional therapeutic substances may also be added to, and held in suspension by, the emollient or cream.

In other aspects, the present invention contemplates methods of making the basic copper-containing therapeutic units, the topical therapeutic formulation containing the units, and the topical mixture containing liposome-encapsulated copper.

Lastly, the present invention contemplates methods of using the basic copper-containing units, the topical mixture containing the units and the topical mixture including the basic units that contain copper as well as other therapeutic substances encapsulated within the volume defined by the bilayer or incorporated into the bilayer or the emollient or cream.

### DETAILED DESCRIPTION

The present invention in its broadest aspect comprises an aqueous solution of copper associated with a carrier that is skin- and tissue-penetrating. This association of copper and the carrier is referred to herein as a “basic therapeutic unit.” The basic therapeutic unit is suitable for topical application to the skin, following which the carrier and the copper pass through the skin and the subjacent tissue so that the copper reaches a position near or at the location of spider veins.

In preferred embodiments, the carrier is a liposome, with a quantity of copper, for example in the form of dissolved copper gluconate, being encapsulated within the volume defined by the membrane or bilayer of each liposome sphere. The liposome/copper association is referred to herein as the “basic liposome therapeutic unit.” The liposomes transport the encapsulated copper through the skin and subjacent tissue to the vicinity of the spider veins.

Liposomes were first produced in 1961 by Alec D. Bangham, who found that phospholipids combined with water immediately formed spherical bodies having the water-encapsulated-in-membrane structure described above. Later work has shown that liposomes may be formed from other substances, such as soy lecithin.

Specific liposomes, after passing through the skin, pass through subjacent tissue and may pass also through the walls of blood vessels or may attach to cellular membranes. Some liposomes appear to fuse with cells, releasing their contents into the cells. Other liposomes are taken up by cells and their membranes are incorporated into the cell membranes, while the encapsulated contents of the liposome are released within the cell. Still other liposomes, are broken down or “digested” by the body tissue through which they pass, ultimately releasing their contents. According to the present invention, the volume defined by the spherical membrane or bilayer of the liposomes encapsulate copper, which is released in the vicinity of spider veins after the liposomes break up.

Where, as here, the goal is to have copper available in the vicinity of the spider veins, the preferred liposomes are those made from soy lecithin. When the soy lecithin is added to a dissolved copper-containing aqueous solution, the liposomes form and encapsulate the aqueous copper solution. The copper-containing liposomes can be topically applied to an area of the skin which overlies spider veins. The liposomes and their encapsulated copper pass through the skin and the subjacent tissue, thereafter residing in the vicinity of the spider veins for alleviation or elimination thereof.

A quantity of the basic liposome therapeutic units may be produced in the following manner. Copper-containing liposomes are produced by dispersing granular soy lecithin and an emulsifier, such as polysorbate 80 in heated (60°-70° C.) sterile water in which some form of copper, such as copper gluconate, has been dissolved, followed by continuous mixing until homogeneous. There is produced a quantity of liposomes which encapsulate the aqueous copper solution.

Approximate exemplary weight percentages of the above ingredients are:

| | |
|---|---|
| copper gluconate | 8.6% |
| lecithin | 12.9% |
| water | 57.1% |
| polysorbate 80 | 21.4% |

While the basic liposome therapeutic units may be topically applied to the skin, liposomes feel oily or greasy and are felt by many to not be pleasing to the touch and to those areas of the skin to which they are topically applied. Thus, the esthetics and “feel” of the basic unit may be improved and rendered acceptable for topical application by producing the therapeutic formulation of the present invention.

The therapeutic formulation is produced by using an emollient or cream to suspend the liposomes. Specifically, the “feel” and cosmetic acceptability of the liposomes may be enhanced by suspending the liposomes in an emollient such as anhydrous vanishing cream to which there may be also added a preservative and a thickener. The vanishing cream and the preservative, for example butylated hydroxytoluene, are heated until a clear melt is obtained. The thickener, which may be xanthan gum, is then added, and mixing is effected until a homogeneous mixture is obtained.

Thereafter, the basic liposome therapeutic formulation and the mixture containing the vanishing cream are admixed while warm and are stirred continuously until room temperature is reached. The resulting therapeutic formulation with the copper-containing liposomes suspended in a vanishing cream is esthetically pleasing and not unpleasant to apply topically to the skin. Moreover, it has been found that the addition of a dispersant, such as simethicone, and a minor amount of peppermint essential oil, render the therapeutic formulation even more pleasing to the touch and smell. Further, an anti-fungal, such as potassium sorbate, and a vasodilator, such as methyl nicotinate may be included. The latter is postulated to cause dilation of the spider veins offering a larger “target,” and more copper receptors, for the copper to beneficially affect.

The approximate weight percentage of the ingredients in the above-described embodiment of the therapeutic formulation is:

| | |
|---|---|
| copper gluconate | 1% |
| vanishing cream | 20% |
| water | 71.1% |
| butylated hydroxytoluene | .1% |
| lecithin | 1.5% |
| xanthan gum | 2% |
| polysorbate 80 | 2.5% |
| simethicone | .5% |
| peppermint oil | .8% |
| potassium sorbate | .2% |
| methyl nicotinate | .3% |

If a greater amount of copper is desired, the copper gluconate percentage may be doubled to 2% by weight and the water decreased to 70.1% by weight.

Clearly, formulations other than those set forth above may be used. The important event is the association of copper with a carrier which acts as a tissue penetration enabler capable of delivering an effective amount of copper directly to the spider veins. As described, one such carrier/enabler, is a liposomal carrier, by which an effective amount of copper can enter the body and pass through the skin and tissue thereof to reach the targeted body sites, specifically locations whereat spider veins are observed.

As noted earlier those having ordinary skill in the liposome art will be able to design and formulate liposomes which can target specific delivery sites. That being the case, copper may, accordingly, be delivered to specified body sites such as those where spider veins are found. Adjusting the amount and type of vanishing cream, thickener, preservative, emulsifier and dispersant, as well as a decision to use or not use peppermint oil or some other similar substance, is within the skill of the art.

Any formulation that is pleasing to the touch and smell and which, when topically applied, allows the copper-carriers to deliver their encapsulated copper for passage through the skin and intervening tissue to reach a spider vein target is within the scope of the present invention and the appended claims. Stated differently, any copper-containing carrier which is capable of acting as a tissue penetrating enabler capable of delivering copper to the site of spider veins following topical application constitutes a basic therapeutic unit or a basic liposome therapeutic unit (both defined above), as set forth in the appended claims, and falls within the scope of the present invention. Whatever else may be added to these basic units, the mere presence of such basic units, with or without more, is sufficient to fall under the umbra of the present invention, as expressed in the claims hereof.

Tests of the above therapeutic formulations have confirmed that topical application thereof to the skin results in both the delivery of copper to subjacent spider vein sites and amelioration or elimination of the spider veins following a period of use. Both skilled in the art and users of the therapeutic formulation are well able to determine when application may be reduced or eliminated simply be observing when the degree of difficulty in discerning the spider veins is acceptable.

Those having skill in the art will appreciate that the main thrusts of this invention arise from the basic therapeutic unit and the basic liposome therapeutic unit, as well as from therapeutic formulations containing same, all as described above and as set forth in the following claims.

What is claimed is:

1. A basic therapeutic unit for topical application to the skin for the alleviation of spider veins in the subjacent tissue, comprising:
a quantity of an aqueous copper solution associated with a carrier which is skin- and tissue-penetration enabler for the solution.

2. A basic therapeutic unit as in claim 1, wherein:
following topical application of the basic therapeutic unit to an area of the skin overlying the spider veins, the carrier penetrates and passes through the skin and subjacent tissue whereupon the copper solution resides in the vicinity of the spider veins.

3. A basic therapeutic unit as in claim 1, wherein:
the carrier is a liposome.

4. A basic therapeutic unit as in claim 1, wherein:
the aqueous copper solution includes a quantity of copper gluconate.

5. A basic therapeutic unit as in claim 4, wherein:
the carrier is a liposome.

6. A therapeutic formulation including the basic therapeutic unit of claim 5, comprising:
a plurality of the basic therapeutic units suspended in a medium which is suitable for topical application to the skin.

7. The therapeutic formulation of claim 5, which further comprises:
a vasodilator in the medium.

8. A basic therapeutic unit suitable for topical application to the skin, comprising:
a quantity of an aqueous solution of copper encapsulated within a liposome.

9. A therapeutic formulation including the basic therapeutic unit of claim 8, which comprises a plurality of the basic therapeutic units in a medium that holds the plural therapeutic units in suspension and is suitable for topical application to the skin.

10. A therapeutic formulation unit of claim 9, which further comprises one or more substances that, in addition to the basic therapeutic units, are held in suspension in the medium.

11. A therapeutic formulation of claim 10, which further comprises:
one or more substances, in addition to copper, that are encapsulated within the liposome.

12. A therapeutic formulation of claim 10, wherein:
a vasodilator is present as an additional substance.

13. A therapeutic formulation of claim 12, wherein:
the vasodilator is methyl nicotinate.

14. A therapeutic formulation of claim 13, wherein:
the liposomes facilitate and enhance the penetration and passage of the basic therapeutic units through the skin and subjacent tissue to permit the copper to arrive at the location of subjacent spider veins, following which the vasodilator enhances the beneficial effects of copper on the spider veins.

15. A therapeutic formulation of claim 14, wherein:
the medium is an emollient or cream.

16. A therapeutic formulation of claim 15, wherein:
the medium is vanishing cream.

17. A therapeutic formulation of claim 7, wherein:
the membrane of the liposome comprises molecules of soy lecithin.

18. A method of making a basic therapeutic unit, which comprises:
encapsulating an aqueous solution of copper within a liposome.

19. A method as in claim 18, wherein:
the copper is in the form of copper gluconate.

20. A method of making a therapeutic formulation from the basic therapeutic unit of claim 19, which comprises:
suspending a plurality of the basic therapeutic units in a medium that is suitable for topical application to the skin.

21. The method of claim 20, wherein:
the medium is an emollient or cream.

22. The method of claim 18, which further comprises:
encapsulating within the liposome substances in addition to copper.

23. The method of claim 18, which further comprises:
suspending in the medium substances in addition to the basic therapeutic units.

24. The method of claim 23, which further comprises:
encapsulating within the liposome substances in addition to copper

25. A method of beneficially affecting the human body by alleviating or eliminating spider veins, which method comprises:
encapsulating an aqueous solution of copper within a liposome; and
applying the liposome to the skin so that the liposome passes through the skin and the subjacent tissue and an effective amount of the copper thereafter resides at or near the spider veins.

26. A method as in claim 25, wherein:
the copper is in the form of copper gluconate

27. The method of claim 26, wherein the liposome-applying step is effected by applying to an area of the skin a medium which is suitable for topical application to the skin and which holds in suspension therein a plurality of copper-encapsulating liposomes.

28. The method of claim 27, wherein a vasodilator is also applied to the area of the skin.

29. The method of claim 28, wherein the vasodilator is held in suspension within the medium.

30. The method of claim 29, wherein the vasodilator is methyl nicotinate.
